(19) United States
(12) Patent Application Publication
(10) Pub. No.: US 2026/0335673 A1
(43) Pub. Date: Oct. 29, 2026

(54) Radiotherapy sheet

(71) Applicant: ALPHA TAU MEDICAL LTD., Jerusalem (IL)

(72) Inventors: Lior Arazi, Tel Aviv (IL); Itzhak Kelson; Amnon Gat, Matan (IL); Ofer Magen, Hod Hasharon (IL); Vered Domankevich, Hod Hasharon (IL); Ronen Segal, Jerusalem (IL); Michael Schmidt, Kfar Saba (IL); Dor Levy, Petach Tikva (IL); Shai Lax, Givat Zeev (IL)

(21) Appl. No.: 19/110,133

(22) PCT Filed: Sep. 10, 2023
(86) PCT No.: PCT/IB2023/058960

§ 371 Date: Mar. 10, 2025

Related U.S. Application Data
(60) Provisional application No. 63/405,441, filed on Sep. 11, 2022.

(51) Int. Cl. A61N 5/10; A61K 51/12

(52) U.S. Cl. CPC A61N 5/1029; A61K 51/1244; A61K 51/1275; A61N 2005/1024; A61N 2005/1085

(57) ABSTRACT

A sheet for radiotherapy, which includes a sheet base configured for placement on tissue of a patient and a radium-binding material coupled to the base sheet. Alpha-emitting radium radionuclides are coupled to the radium-binding material. The radium radionuclides do not leave the sheet, but radon and lead resulting from decay of the radium radionuclides easily leave the sheet.

### FIELD OF THE INVENTION

The present invention relates generally to radiotherapy and particularly to sources carrying particles for radiotherapy.

### BACKGROUND

Ionizing radiation is commonly used in treatment of certain types of tumors, including malignant cancerous tumors, to destroy cells of the tumors. Various methods have been used to deliver the ionizing radiation to the tumor. One of these methods is implantation of sources (commonly referred to as “seeds”), encapsulating radiation-emitting atoms also referred to as radionuclides, in or near the tumor. The accurate placement of a plurality of sources is not a simple task and it has been suggested to attach a plurality of sources to be placed together.

U.S. Pat. No. 4,754,745 to Horowitz describes a conformable sheet of material which is absorbable in living tissue, having a plurality of radioactive sources implanted in a predetermined array in the sheet.

U.S. Pat. No. 5,030,195 to Nardi describes a non-absorbable seed patch which has radioactive seeds threaded therein.

U.S. Pat. No. 6,099,457 to Good, titled: “Endocurietherapy” describes a plurality of radiation emitters spaced throughout a fabric base.

US patent publication 2003/0092957 to Scott et al., describes a brachytherapy device comprising a substrate having a radioactive coating layer formed thereon. Additionally, this reference describes radioactive sol-gels having an insoluble radioisotope dispersed therein and an expandable implant containing ribs for radioactive rods. Radioisotopes are embedded in the surface to avoid delamination.

US patent publication 2004/0076579 to Coniglione et al. describes a film formed in a single step, such as by extrusion or molding from a radioactive composite containing radioactive particles. PCT publication WO2017/200298 describes placing a liquid radioactive isotope at a constant thickness on an irregular skin surface for treating skin cancer.

US patent publication 2014/0296609 to Desantis et al. describes a layer structure for epidermal radionuclide therapy.

U.S. Pat. No. 9,022,915 to Nakaji describes radioactive seeds embedded in respective radioactive seed carriers.

Alpha particles are a powerful means for radiotherapy since they create complex DNA damage that is difficult to repair and are effective also against cells which show high resistance to other types of radiation. Moreover, alpha particles have a short range, such that when an alpha-emitting atom is brought to the immediate vicinity of a cancer cell, the emitted alpha particle has a high chance of killing the cell while sparing surrounding healthy tissue.

Diffusing alpha-emitters radiation therapy (DaRT), described for example in U.S. Pat. No. 8,834,837 to Kelson et al., extends the therapeutic range of alpha radiation, by using radium-223 or radium-224 atoms, which have within a relatively short time span a chain of several radioactive decays. In DaRT, the radionuclides are attached to a source implanted in the tumor with sufficient strength such that the radionuclides do not leave the source in a manner that they go to waste, but a substantial percentage of the daughter radionuclides leave the source into the tumor, upon decay. These daughter radionuclides spread by diffusion and possibly also convection over a region measuring several millimeters in size. Thus, the range of destruction in the tumor is increased relative to sources loaded with radionuclides which remain with their daughters on the source. An advantage of DaRT is the ability to destroy tumors using sources having low levels of radioactivity. The effective range of DaRT sources is limited to several millimeters and therefore when treating a large tumor, there may be a need to implant in the tumor several dozens or even hundreds of sources to ensure destruction of the entire tumor volume. In addition, to avoid cold spots, the sources need to be placed at specific relative angles and locations. Implanting such a large number of sources is a tedious task which may take a significant amount of time.

Lior Arazi, “Diffusing Alpha-Emitters Radiation Therapy: Theoretical and Experimental Dosimetry”, Thesis submitted to the senate of Tel Aviv University, September 2008, the disclosure of which is incorporated herein by reference, describes use of a plaque loaded with radium-224 on its surface to irradiate a wall of a surgically formed cavity following tumor resection. The planar source is described as carrying an activity of 1 or 3μCi/cm².

### SUMMARY

There is therefore provided in accordance with an embodiment of the present invention, a sheet for radiotherapy, including a sheet base configured for placement on tissue of a patient, a radium-binding material coupled to the base sheet; and alpha-emitting radium radionuclides coupled to the radium-binding material, wherein the radium radionuclides do not leave the sheet, but radon and lead resulting from decay of the radium radionuclides, easily leave the sheet.

Optionally, the sheet base is formed from the radium-binding material. Optionally, the sheet base is formed of a material different from the radium-binding material. Optionally, the radium-binding material is coated over the sheet base. Optionally, the radium-binding material comprises mini-particles, micro-particles and/or nano-particles dispersed on the sheet base. Optionally, the alpha-emitting radium radionuclides have an activity of at least 1 microcurie per centimeter length. Optionally, the radium-binding material comprises manganese oxide. Optionally, the radium-binding material comprises alginate. Optionally, the sheet base comprises a silicone substrate. Optionally, the sheet base comprises a mesh. Optionally, the mesh comprises a titanium mesh.

There is further provided in accordance with an embodiment of the present invention, a sheet for radiotherapy, including a sheet-base configured for placement on tissue of a patient; and sources of a length of at least one millimeter, carrying alpha-emitting radionuclides with an activity of at least 1 microcurie per centimeter length, coupled to the sheet base, such that at least part of the sources are external to the sheet base. Optionally, the sheet base is configured to mold to contours of the skin of the patient. Optionally, the sheet base comprises a silicone sheet. Optionally, the sheet base comprises a mesh and the sources have internal tunnels threaded on the mesh. Optionally, the sheet base has an area of at least 2 centimeters square. Optionally, the apparatus includes a layer of gel on the sheet. Optionally, the alpha-emitting radionuclides comprise radium-224 radionuclides. Optionally, the alpha-emitting radionuclides are coupled to the sources in a manner which prevents detachment of the alpha-emitting radionuclides from the sources but allows escape from the sources of daughter radionuclides of the alpha-emitting radionuclides, upon decay. Optionally, the alpha-emitting radionuclides have an activity of at least 5 microcurie per centimeter length. Optionally, the sources protrude from the sheet base by at least 1 millimeter. Optionally, the sources are coupled to the sheet base by an adhesive.

There is further provided in accordance with an embodiment of the present invention, an apparatus for radiotherapy, comprising a plurality of sources, each having a length of at least one millimeter, carrying alpha-emitting radionuclides, coupled to each other in a manner forming a sheet suitable for placement on patient tissue. The plurality of sources cover at least 50% of an area of the sheet. Optionally, the sources have an internal tunnel and are coupled to each other by one or more threads which pass through the internal tunnels of the sources. Optionally, the plurality of sources cover at least 90% of an area of the sheet. Optionally, the plurality of sources each have a length of at least 5 millimeters.

There is further provided in accordance with an embodiment of the present invention, a method of radiotherapy, comprising covering a tumor on the skin of a patient with a gel, providing a sheet to which radium-224 radionuclides at a density of an activity of at least 1 microcurie per square centimeter are coupled; and placing the sheet on the gel for a duration suitable for radiotherapy.

Optionally, the radium-224 radionuclides have an activity of at least 10 microcurie per square centimeter. Optionally, the gel has a thickness of less than 0.6 mm. Optionally, the gel has a thickness of less than 50 microns. Optionally, covering the tumor with the gel comprises placing gel on the sheet and placing the sheet, with the gel thereon, on the skin of the patient. Optionally, placing the sheet on the gel is performed after the tumor is covered by the gel. Optionally, the method includes identifying contours of the tumor and wherein providing the sheet comprises coupling radium-224 radionuclides to the sheet in an area selected responsive to the identified contours, before it is placed on the gel. Optionally, the method includes identifying contours of the tumor and wherein placing the sheet on the gel comprises placing a mask following the contours of the tumor around the tumor and placing the sheet on the gel and the mask. Optionally, the gel does not contain alpha-emitting radionuclides.

There is further provided in accordance with an embodiment of the present invention, an apparatus for radiotherapy, comprising a sheet configured for placement on a surface of a patient; and radium-224 radionuclides coupled to the sheet at a density of an activity of at least 10 microcurie per square centimeter.

### BRIEF DESCRIPTION OF THE DRAWINGS

FIG. 1A is a schematic illustration of a radiotherapy sheet including embedded cylindrical sources, in accordance with an embodiment of the present invention;

FIG. 1B is a cross-section of the radiotherapy sheet of FIG. 1A;

FIG. 1C is a schematic illustration of a radioactive sheet, in accordance with another embodiment of the invention;

FIG. 2A is a schematic illustration of a radiotherapy sheet formed from sources coupled to each other, in accordance with an embodiment of the present invention;

FIG. 2B is a schematic illustration of a radiotherapy sheet formed from sources coupled to each other, in accordance with another embodiment of the present invention;

FIG. 2C is a cross section of a radiotherapy sheet, formed of sources, in accordance with another embodiment of the invention;

FIG. 2D is a cross section of a radiotherapy sheet, formed of sources, in accordance with still another embodiment of the invention;

FIG. 3A is a schematic illustration of a radiotherapy sheet, in accordance with an embodiment of the present invention;

FIG. 3B is a schematic cross section view of the radioactive sheet of FIG. 3A.

FIG. 4A is a schematic cross-section of a radioactive sheet, in accordance with another embodiment of the invention;

FIG. 4B is a schematic cross-section of a radioactive sheet, in accordance with another embodiment of the invention; and

FIG. 5 is a flowchart of a method for treating a tumor with a radioactive sheet, in accordance with an embodiment of the present invention.

### DETAILED DESCRIPTION OF EMBODIMENTS

An object of aspects of some embodiments of the invention is to provide a sheet for radiotherapy treatment which includes radium-224 coupled to the sheet in a manner that the radium does not prematurely leave the sheet, but daughter radionuclides leave the sheet upon radioactive decay. The radium-224 is optionally coupled to the sheet in a manner such that its daughter radionuclides have a desorption probability to leave the sheet into an adjacent tumor, of at least 10%, at least 30%, at least 50%, at least 70% or even at least 80%. This object is achieved, in some embodiments, by including in the sheet an intermediary, which couples to the radium, on the one hand and is fixed to the sheet, on the other hand.

In order to prevent premature release of the radium, the radium is optionally attached to carriers at least partially embedded in the sheet. In some embodiments, the carriers are mini-particles, micro-particles or even nanoparticles. In other embodiments, the radium-224 is mounted on sources (commonly referred to as seeds) which are partially embedded in the sheet or mounted on a surface of the sheet. The sources are optionally placed on the sheet in a manner such that the sheet does not come between the sources and the patient tissue. In still other embodiments, the sheet itself is formed predominantly of radioactive sources.

The sheet is optionally adapted for placement externally on the skin of a patient, including a tumor. The sheet is optionally used for treatment of skin cancer (e.g., basal-cell carcinoma (BCC) or squamous cell carcinoma (SCC)) or a pre-cancer tumor. In other embodiments, the sheet is adapted for placement on a wall of a cavity formed by surgical removal of a tumor. In still other embodiments, the sheet is placed on a cancer tumor surrounding critical body tissue, such as a main blood vessel, which cannot be removed or damaged in surgery.

An aspect of some embodiments of the invention relates to a radioactive sheet comprising a base substrate which provides strength preventing disintegration, on which a radium-binding material is placed along with radium radionuclides which emit alpha-radiation and daughter radionuclides. The base substrate and radium-binding material are configured such that radium does not leave the sheet in substantial amounts, while daughter radionuclides are allowed to leave the sheet into the tumor.

In some embodiments, the base substrate is formed of a material different from the radium-binding material. In other embodiments, the same material is used to form the base substrate and to bind radium. Optionally, the base substrate comprises both a continuous surface and a mesh which reinforces the sheet. Alternatively or additionally, the radium-binding material is provided as a mesh which carries radium.

An aspect of some embodiments of the invention relates to a radioactive sheet, formed in a major extent from radium-carrying sources, which have a length greater than 1 millimeter. Optionally, at least 30% of the weight of the sheet, at least 40% of its weight, at least 50% of its weight, at least 60% of its weight, at least 70% of its weight, at least 80% of its weight or even at least 90% of its weight results from the radium-carrying sources. Alternatively or additionally, at least 30% of the area of the sheet, at least 40% of its area, at least 50% of its area, at least 60% of its area, at least 70% of its area, at least 80% of its area or even at least 90% of its area is covered by the radium-carrying sources. In some embodiments, the radium-carrying sources are cylindrical and have a length of at least 2 millimeters, or even at least 5 millimeters. Optionally, the radium-carrying sources are metallic. Alternatively or additionally, the radium-carrying sources are made of a material different than the material used to combine the sources into a sheet.

In some embodiments, the radioactive sheet is formed of the radium-carrying sources, such that absent the radium-carrying sources the sheet would not exist (e.g., would disintegrate into many pieces). Alternatively, the radioactive sheet is formed of a mesh on which the radium-carrying sources are mounted. Optionally, in this alternative, the area not covered by the radium-carrying sources is less than 40%, less than 20% or even less than 10% of the total area covered by the sheet.

An aspect of some embodiments of the invention relates to a radioactive sheet, which comprises a base and a plurality of cylindrical radium-carrying sources partially mounted on the base in a manner that at least a portion of the cylindrical sources bulge from the sheet. The base of the sheet is optionally flexible allowing the sheet to conform to patient tissue. In some embodiments, the base of the sheet comprises a mesh. In other embodiments, the base comprises a continuous layer formed, for example, from silicone or a polymer.

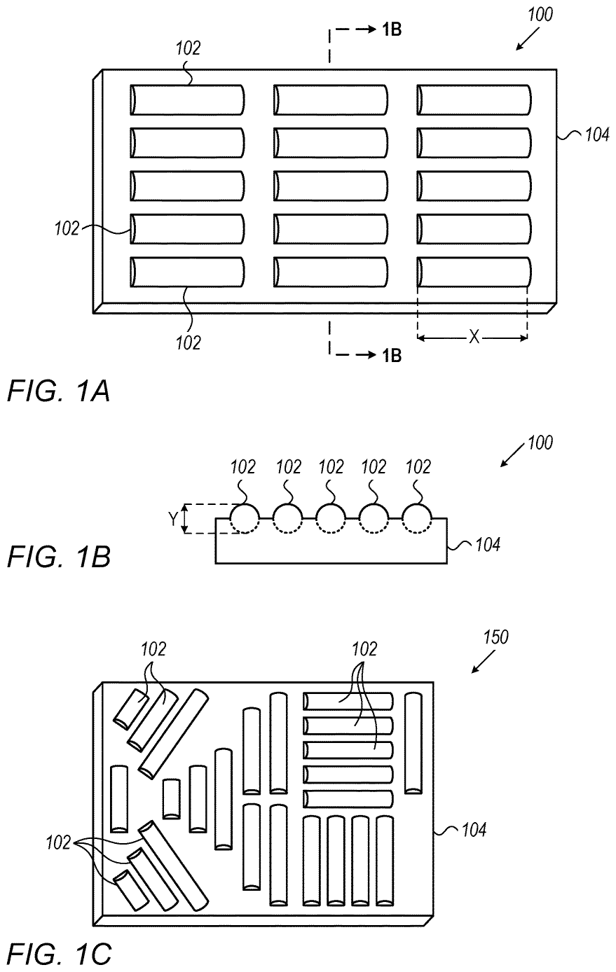

FIG. 1A is a schematic illustration of a radiotherapy sheet 100 carrying sources 102 on a base substrate 104, in accordance with an embodiment of the invention. FIG. 1B is a cross-section view of radiotherapy sheet 100.

Base substrate 104 comprises a flexible biocompatible material which allows manipulation and placement on a surface of the tumor, in a manner which conforms to the tumor, so that the radionuclides on sources 102 are adjacent the tumor. Base substrate 104 is optionally of sufficient strength to preserve its integrity at least until implantation or until most of the activity on the sheet has been expended.

Suitable materials for base substrate 104 include, for example, polymer, rubber, textile, plastic, metal (e.g., stainless steel, titanium mesh) or a combination of materials. Preferably, sheet 100 is easily moldable to the contours of the tumor surface. Base substrate 104 optionally has a low biodegradability, of less than a nanometer of the sheet thickness per day, or even less than 0.5 nanometers per day. In some embodiments, base substrate 104 is not biodegradable at all. Alternatively, base substrate 104 has a high biodegradability, such that it biodegrades entirely within less than 6 months, less than a month, less than a week, less than a day or even less than 8 hours. Optionally, base substrate 104 is made of materials and a sufficient thickness such that alpha particles leaving sources 102 do not pass through base substrate 104 to leave the sheet 100 from the opposite face of the sheet. In some embodiments, base substrate 104 comprises a gauze pad, for example a Paraffin gauze. In other embodiments, base substrate 104 comprises an alginate wound dressing pad or a poloxamer (e.g., pluronics) pad.

In some embodiments, base substrate 104 has a thickness of at least 0.1 millimeters, at least 0.3 millimeters, at least 0.5 millimeters, at least 0.7 millimeters or even at least 0.9 millimeters. Optionally, base substrate 104 has a thickness of less than 6 millimeters, less than 5 millimeters, less than 4 millimeters or even less than 3 millimeters.

In some embodiments, sources 102 are cylindrical and have a length X of at least 2 millimeters, at least 5 millimeters or even at least 10 millimeters. Optionally, sources 102 have a length which is smaller than 70 mm, smaller than 60 mm or even smaller than 40 mm (millimeters). Sources 102 optionally have a diameter Y of 0.7-1 mm, although in some cases, sources of larger or smaller diameters are used. Particularly, for treatment layouts of small spacings, sources 102 optionally have a diameter of less than 0.7 mm, less than 0.5 mm, less than 0.4 mm or even not more than 0.3 mm.

Sources 102 are optionally made of a material which binds to radium, for example an electrically-conductive metal, such as stainless steel and/or titanium. Alternatively, sources 102 comprise a non-metallic rod from a material or mixture of materials which includes a substantial amount of a non-metallic material which binds to radium, such as alginate or poloxamer. Further alternatively, sources 102 comprise a non-metallic material, such as a polymer and/or silicone, in which a metallic material which binds to radium, such as manganese oxide, is dispersed. Alternatively or additionally, sources 102 comprise a material or mixture of materials which does not bind to radium, but are coated by a radium-binding coating. Sources 102 may be generated, for example, using any of the methods described in U.S. Pat. No. 8,894,969 to Kelson et al., which is incorporated herein by reference, although other methods of production may be used. In some embodiments, sources 102 are made at least partially of a relatively flexible material, such as gold, polymer and/or silicone, in order to avoid adding unnecessary rigidity to sheet 100. Sources 102 are hollow, in some embodiments, to allow passage of sutures to pass through the sources 102. In other embodiments, sources 102 are solid (i.e., not hollow).

Sources 102 are optionally partially embedded in base substrate 104, in order to strengthen the bond between base substrate 104 and sources 102. In some embodiments, at least 20% of the thickness of sources 102, at least 35% of their thickness, at least 60% of their thickness, or even at least 80% of the thickness is embedded in base substrate 104. On the other hand, in order to prevent base substrate 104 from blocking radionuclides from reaching the tumor, less than 90%, less than 80%, less than 70% or even less than 60% of the thickness of sources 102 is embedded in base substrate 104. In embodiments in which sources 102 are cylindrical, at least 30°, at least 60°, at least 90°, or even at least 120°of the circumference of the sources are outside of base substrate 104. In some embodiments, sources 102 extend beyond base substrate 104 by at least 0.1 millimeter, at least 0.2 millimeters or even at least 0.3 millimeters.

In some embodiments, sources 102 are coupled to base substrate 104 by an adhesive (e.g., MED 4213, MED-6033 and/or MED-6233). Alternatively or additionally, base substrate 104 is produced with slots sized and shaped to tightly receive the sources 102. In some embodiments, for example when base substrate 104 comprises a silicone base, sources 102 are bonded to base substrate 104 by heating radiotherapy sheet 100. Further alternatively or additionally, sources 102 are tied to base substrate 104 using a suitable suture. Optionally, in accordance with this alternative, sources 102 are hollow and the suture is passed through the source and tied to the base substrate 104.

In some embodiments, base substrate 104 comprises a mesh made of a medical thread, for example a knitted mesh, and sources 102 are fitted on the thread at suitable locations of the mesh. Optionally, not all of sheet 100 is covered by sources 102, to allow for sufficient flexibility of sheet 100. Particularly, the rim of sheet 100 is optionally free of sources 102 to allow simple fixation of sheet 100 to a patient. In some embodiments, the surface area of sheet 100 covered by sources 102 is at least 20%, at least 30%, at least 40% or even at least 60% of the surface area of sheet 100. Optionally, the surface area of sheet 100 covered by sources 102 is less than 90%, less than 80% or even less than 60% of the surface area of sheet 100. Adjacent sources 102 are optionally separated from each other by at least 0.1 millimeters, at least 0.3 millimeters or even at least 0.5 millimeters. On the other hand, the distance between adjacent sources 102 is optionally smaller than 1.5 millimeters, smaller than 1.2 millimeters, or even smaller than 1 millimeter.

As shown, sources 102 are all placed in sheet 100 in the same direction. In other embodiments, sources 102 are placed in different directions in sheet 100.

FIG. 1C is a schematic illustration of a radioactive sheet 150, in accordance with another embodiment of the invention. Sheet 150 illustrates the use of partially embedded sources 102 of different lengths, as well as irregular arrangement of the sources, including diagonal placement. Such irregular arrangements may be used, for example, in cases in which the sheet is intended to be folded around a tumor in a three-dimensional folding.

Sources 102 carry radionuclides which provide therapeutic doses of radiation. Typically, the radionuclide, its daughter radionuclides, and/or its subsequent nuclei in the decay chain are alpha-emitting, in that an alpha particle is emitted upon the decay of any given nucleus. For example, the radionuclide may comprise an isotope of Radium (e.g., Ra-224 or Ra-223), which decays by alpha emission to produce a daughter isotope of Radon (e.g., Rn-220 or Rn-219), which decays by alpha emission to produce an isotope of Polonium (e.g., Po-216 or Po-215), which decays by alpha emission to produce an isotope of Lead (e.g., Pb-212 or Pb-211), as described, for example, in U.S. Pat. No. 8,894,969, which is incorporated herein by reference. Alternatively, the radionuclide comprises Actinium-225.

Sources 102 optionally have an activity of at least 1 microcurie per centimeter length, at least 3 microcurie per centimeter length, at least 5 microcurie per centimeter length, or even at least 10 microcurie per centimeter length. The radionuclides are optionally coupled to sources 102 in a manner such that daughter radionuclides have a desorption probability of at least 20%, at least 35%, at least 45%, at least 60% or even at least 70% to leave the sources. Alternatively, sources 102 have a low desorption probability of less than 30%, less than 25% or even less than 20%, in order to increase the effect of beta radiation relative to the alpha radiation. In accordance with this alternative, sources 102 optionally have a relatively high activity, of at least 6 microcurie per centimeter length.

The radionuclides of the sources 102 may be dispersed evenly around the circumference of the sources 102 for simplicity of production, or may be concentrated mainly (at least 60%), predominantly (at least 80%) or entirely (at least 99%) in the area not embedded in base substrate 104.

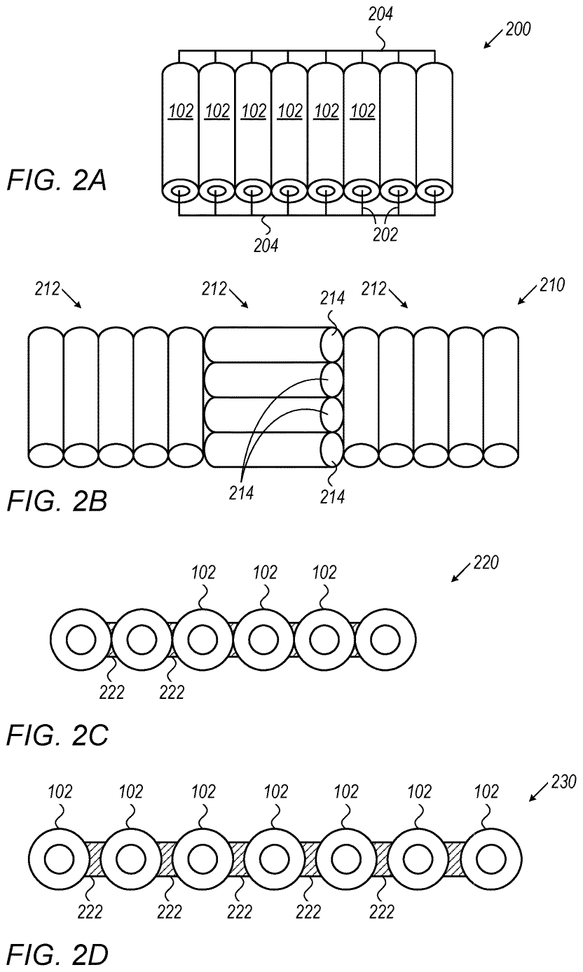

FIG. 2A is a schematic illustration of a radiotherapy sheet 200, formed of sources 102, in accordance with an embodiment of the invention. Sheet 200 is formed of a plurality of cylindrical sources 102 carrying radium radionuclides, coupled to each other. In FIG. 2A, sources 102 are arranged parallel to each other in sheet 200. Medical threads 202 are passed through each of sources 202 and are tied on both ends to external threads 204 in a manner which holds sources 102 together, forming sheet 200.

FIG. 2B is a schematic illustration of a radiotherapy sheet 210, formed of sources 102, in accordance with another embodiment of the invention. Sheet 200 comprises a plurality of patches 212 of parallel sources 102. The patches 212 are connected to each other with the ends 214 of their sources 102 connected to the length of a source of another patch.

It is noted that FIGS. 2A and 2B are examples, and other arrangements of sources may be used to form sheets in accordance with embodiments of the present invention.

FIG. 2C is a cross section of a radiotherapy sheet 220, formed of sources 102, in accordance with another embodiment of the invention. Sources 102 are optionally attached using an adhesive 222 (e.g., MED 4213 and/or MED-6033). In some embodiments in which sources 102 are cylindrical, the sources 102 are placed adjacent each other and the adhesive 222 is placed near the upper and lower surfaces of the sheet, where sources 102 do not touch each other.

FIG. 2D is a cross section of a radiotherapy sheet 230, formed of sources 102, in accordance with still another embodiment of the invention. Sheet 230 differs from sheet 220 of FIG. 2C in that the adhesive is spread throughout the entire thickness of sheet 230. Sheet 230 has a lower area of the sheet covered by sources 102 relative to sheet 220, and may be used to provide more flexibility in cases when flexibility is more important than coverage. In other embodiments, sources 102 are connected to each other by soldering. Soldering may be used, for example, when the sheet is relatively small, and flexibility is less of an issue.

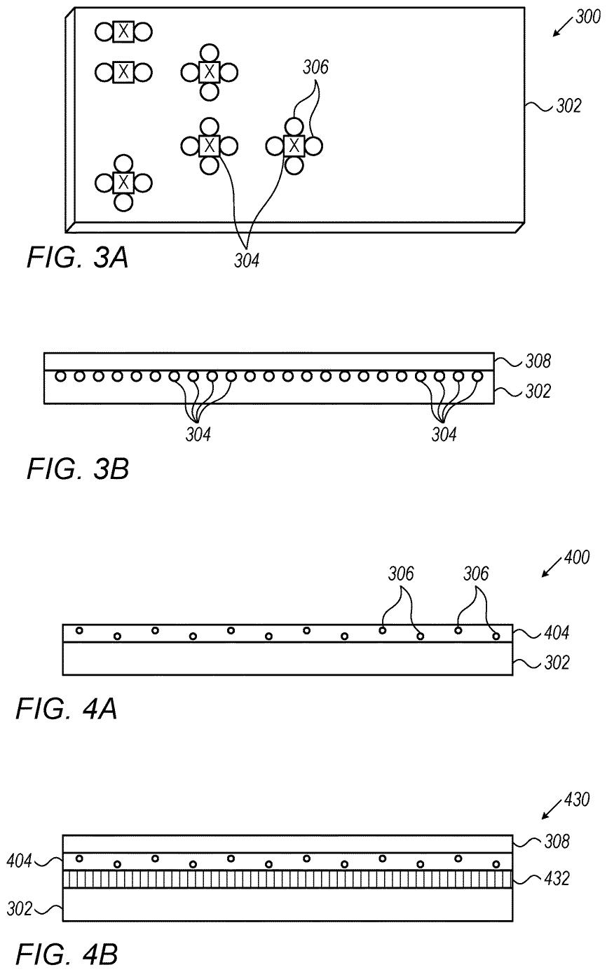

FIG. 3A is a schematic illustration of a radioactive sheet 300, in accordance with another embodiment of the invention. FIG. 3B is a schematic cross section view of radioactive sheet 300 of FIG. 3A. Sheet 300 comprises, in some embodiments, a base substrate 302 formed of a flexible material which does not strongly bind to radium, such as for example, polymer, rubber, textile, and/or plastic. In order to hold the radium, radium-binding elements 304 are placed on the surface, or very close to the surface, of base substrate 302 and radium radionuclides 306 are coupled to the radium-binding elements 304. It is emphasized that FIGS. 3A and 3B are not drawn to scale and radionuclides 306 and possibly also radium-binding elements 304 are shown much larger than they actually are, so that they can be seen in FIG. 3A. Radium-binding elements 304 decrease the diffusion length of the radium in sheet 300, preventing the radium from leaving the sheet or diffusing in the sheet away from the tumor.

Base substrate 302 is optionally of a similar structure to that of base substrate 104 and may have the structure of any of the embodiments discussed above. In some embodiments, base substrate 302 comprises a material permeable to radon, such as silicone (e.g., Polydimethylsiloxane (PDMS)). Thus, radon entering the base substrate is allowed to diffuse out of the base substrate 104 into a tumor being treated. Optionally, base substrate 104 comprises a material also permeable to lead, allowing lead generated in the substrate to diffuse out of the substrate into the tumor.

Radium binding elements 304 may bind to radium due to the material they are formed of (e.g., manganese oxide, alginate) and/or due to their structure (e.g., microparticles, nanoparticles). In some embodiments, radium-binding elements 304 comprise manganese dioxide. Optionally, radium-binding elements 304 are placed on base substrate 302 as a coating. In some embodiments, the radium-binding elements 304 are loaded with radium before being coated onto the base substrate 302. In other embodiments, radium radionuclides 306 are added to the radium-binding elements 304 after the radium-binding elements 304 are coated onto the base substrate 302. In some embodiments, a thin protective layer 308 is added above the radium-binding elements 304 coating, to prevent escape of the radium-binding elements 304 from the sheet 300. The protective layer 308 optionally comprises a material which prevents release of radium, but is permeable to daughter radionuclides of radium, particularly radon and lead. Optionally, protective layer 308 comprises a silicone, such as PDMS, optionally a NUSIL PDMS.

Optionally, in these embodiments, protective layer 308 has a thickness of less than 5 micrometers, less than 1 micrometer, less than 0.1 micrometers, such that radium-binding elements 304 are within less than 50 nanometers, or even less than 20 nanometers from the surface of sheet 300.

In other embodiments, protective layer 308 is a removeable layer, which is removed from sheet 300 shortly before the sheet is applied to the tumor. In these embodiments, protective layer 308 is not necessarily thin and/or is not formed of a material permeable to the daughter radionuclides.

Radium-binding elements 304 include, in some embodiments, mini-particles, having a largest dimension of more than 0.05 millimeters, but less than 0.5 millimeters, or even less than 0.1 millimeters. In embodiments requiring that sheet 300 be flexible, for example when sheet 300 is relatively large, the density of mini-particles is selected to not hinder the flexibility of the sheet. In other embodiments, the radium-binding elements 304 are microparticles, having a largest dimension of more than 0.05 micrometers, but less than 250 micrometers, less than 150 micrometers or even less than 100 micrometers. In one embodiment, the microparticles have a largest dimension of about 50 micrometers (e.g., 50 microns +20%).

In still other embodiments, the radium-binding elements 304 are nanoparticles, having a largest dimension not greater than 50 nanometers, or even less than 10 nanometers. The nanoparticles include, for example, gold particles and/or titanium particles, which are easily visible by imaging modalities. Alternatively, the nanoparticles are made of alginate or poloxamer.

Alternatively to placing the radium-binding elements 304 and the radium radionuclides 306 on the surface of base substrate 302, base substrate 302 is biodegradable and the radium-binding elements 304 and the radium radionuclides 306 are embedded in base substrate 302. As base substrate 302 degrades, the radium 306 and/or its daughter radionuclides are released into the tumor.

In other embodiments, base substrate 302 comprises a metal (e.g., stainless steel, titanium mesh). In some of these embodiments, radium-binding elements 304 are not used and the radium is fixed directly to base substrate 302. The fixation of the radium to base substrate 302 may be performed using any suitable method known in the art, such as electrically charging base substrate 302 in a flux of radium from a thorium source.

Optionally, sheet 300 has an active area carrying radium radionuclides of at least 0.5 cm square, at least 1 cm square, at least 2 centimeters square or even at least 4 centimeters square, at least 10 centimeters square, or even at least 20 centimeters square. The sheet optionally has margins of at least 1 millimeter or even at least 2 millimeters, which do not carry radium radionuclides.

The radium-224 optionally has an activity of at least 1 microcurie per square centimeter, at least 3 microcurie per square centimeter, at least 5 microcurie per square centimeter, at least 10 microcurie per square centimeter, at least 20 microcurie per square centimeter, at least 50 microcurie per square centimeter or even at least 100 microcurie per square centimeter.

FIG. 4A is a schematic cross-section of a radioactive sheet 400, in accordance with another embodiment of the invention. Sheet 400 differs from sheet 300 in that rather than using radium-binding elements 304 to bind the radium, sheet 400 includes a radium-binding layer 404 in which radium radionuclides 306 are embedded.

In some embodiments, the radium-binding layer 404 comprises alginate and/or poloxamer, which bind radium and thus prevent the radium radionuclides 306 from leaving sheet 400, while allowing daughter radionuclides to leave sheet 400 upon decay, and enter a tumor on which radioactive sheet 400 is placed. The alginate or poloxamer layer is optionally provided as a mesh or continuous surface. Optionally, calcium is added to the radium-binding layer 404 in an amount selected to control its flexibility. In some embodiments, both base substrate 302 and radium-based layer 404 are formed of alginate and sufficient calcium is included therein to ensure the integrity of sheet 400. Optionally, in this embodiment, base substrate 302 has a higher concentration of calcium than radium-binding layer 404. In other embodiments, base substrate 302 comprises a mesh or pad, which absorbs a significant percentage of the alginate, when the alginate is placed on base substrate 302.

In one embodiment of production of radioactive sheet 400, the production begins with a base substrate, e.g., a silicone substrate, serving as base substrate 302. A layer of a radium-binding material, such as manganese oxide, to serve as radium-binding layer 404, is coated onto the base. Thereafter, liquid radium-224, of an amount of a desired activity, is loaded on the manganese oxide. A thin protective layer 406 is added on the radium-binding layer 404 and the radium coupled to it, to prevent release of the radium, while still allowing daughter radionuclides, e.g., radon and/or lead, to leave sheet 400 into the tumor. Optionally, the amount of the radium-binding material in radium-binding layer 404 is selected to be suitable for coupling to significantly more radium than the amount required to provide the desired activity, such that the activity is set by the aunt of radium used. Alternatively, the amount of the radium-binding material in radium-binding layer 404 is selected to allow for up to the amount of radium providing the desired activity, such that even if more radium is placed on the sheet during production, only the radium with the desired activity will remain on the sheet.

Alternatively or additionally, radium-binding layer 404 comprises a semi-rigid sol-gel sheet, with the radium-224 within the gel.

FIG. 4B is a schematic cross-section of a radioactive sheet 430, in accordance with another embodiment of the invention. Radioactive sheet 430 is similar to sheet 400, but additionally includes a mesh 432 on base substrate 302. Mesh 432 optionally adds strength to the base substrate 302, to prevent undesired ripping. Alternatively or additionally, mesh 432 provides a stronger bind to radium-binding layer 404 (e.g., to manganese oxide) and/or to the radium. Mesh 432 optionally comprises a metal, such as titanium.

In one embodiment of production of radioactive sheet 430, the production begins with dipping mesh 432 into a manganese oxide or alginate solution to form a radium-binding coating on the mesh. Thereafter, the mesh is dipped into a radium solution. The mesh 432, with the radium is optionally attached to base substrate 302. In some embodiments, after attaching mesh 432 to base substrate 302, the mesh is coated by a protective layer which prevents the radium from leaving the sheet 430. It is noted that this method is brought by way of example and the acts of the method may be performed in a different order, or other production methods may be used. For example, instead of loading the mesh with radium before attaching the mesh 432 to the base substrate 302, the mesh 432 is first attached to base substrate 302 and then the radium is coupled to the mesh 432. In some embodiments, mesh 432 is not coated by a radium-binding material, and instead radium is coupled directly to the mesh, for example using electrical charge and/or heat treatment.

It is further noted that in some embodiments the mesh is not attached to a base substrate at all, but rather is sufficiently strong to serve as a base of the sheet on its own. In other embodiments, rather than being placed on base substrate 302, mesh 432 is embedded in base substrate 302.

In some embodiments, the radium is attached to the sheet on its own, in a free state, and measures are taken to ensure that the sheet is as close as possible to the tumor, so that a large share of the radium and/or its daughter radionuclides reach the tumor. The term free refers to the radionuclides as not coupled to large particles (e.g., particles having a diameter greater than 100 nanometers) which interfere with flow between cells, not coupled to targeting elements (e.g., antibodies) which couple to cells, and not coupled to vectors which are internalized into cancer cells (e.g., liposomes, radionucleosides).

The above-described sheets may be provided in various sizes and shapes, including rectangular and circular. In some embodiments, the sheet has a square shape with a size of at least 10 millimeters, for example 12×12 millimeters or 23×23 millimeters. In another embodiment, the sheet is circular with a diameter of between 10-20 millimeters, for example 15 millimeters.

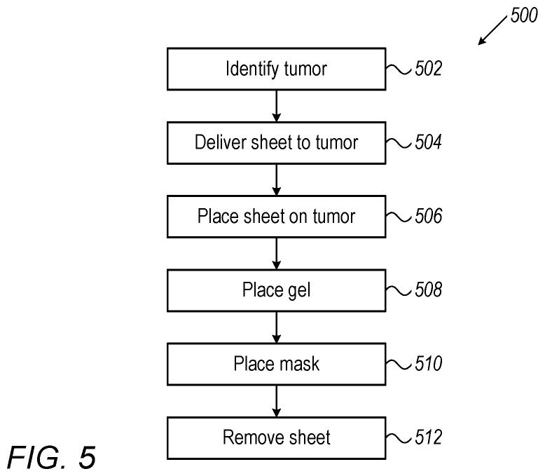

FIG. 5 is a flowchart of a method 500 for treating a tumor with a radioactive sheet, in accordance with an embodiment of the present invention. Method 500 starts with identifying (502) a tumor which can benefit from alpha-emitter radiotherapy provided by a radioactive sheet. The radioactive sheet is delivered (504) to the identified tumor and placed (506) on the tumor. In some embodiments, a hydrophobic material, which allows daughter radionuclides, particularly radon and lead, to diffuse through it, is placed (508) on the tumor, between the tumor and the sheet. The hydrophobic material includes, in some embodiments, a gel. Optionally, the hydrophobic material does not include alpha-emitting radionuclides, and possibly does not include radionuclides at all, The hydrophobic material is optionally used to enhance the coupling of the sheet to the patient tissue, and prevent daughter radionuclides of radon resulting from radium decal, from escaping into the air instead of entering the tumor.

In some embodiments, a protective mask having a solid frame and an internal hole matching the contours of the tumor to be treated, is placed (510) between the patient and the radioactive sheet, to protect tissue of the patient surrounding the tumor, which is not to be damaged by the radiation. In some embodiments, the sheet is removed (512) after a treatment period.

In some embodiments, after placing (506) the radioactive sheet on the tumor, the radioactive sheet is fixated to the tumor, for example, by a suture, bandage and/or adhesive. Optionally, the radioactive sheet is fastened by one or more elastic bands which continuously pushes the radioactive sheet against the tumor, so that after a first layer of the tumor is destroyed, the sheet moves closer to the current outer layer of the tumor. The elastic bands optionally comprise Nylon and/or rubber bands.

As to delivering (504) the sheet, when used for treatment of an internal tumor, the sheet may be delivered to the tumor during surgery for removal of a tumor and placed in the cavity of the removed tumor to kill residual cancerous cells. In some embodiments, the sheet is delivered to the tumor rolled up or folded, through an endoscope, catheter or other minimally invasive tool.

Referring in more detail to placing (508) the gel, in some embodiments that sheet is provided with a thin layer of gel, on the surface of the sheet to be placed on the tumor. It is noted that in these embodiments, when the sheet is placed on skin of the patient, gel may cover the sheet also in areas which do not contact the tumor. Alternatively or additionally, gel is provided separately from the sheet to enhance contact between the sheet and the tumor. The gel layer is optionally very thin, for example having a thickness of less than 0.6 mm, less than 0.1 mm, less than 0.05 mm, or even less than 20 microns. The gel is optionally configured to prevent Radon resulting from Radium Decay from evaporating or otherwise escaping to a distance from the tumor at which further decay of the Radon or daughter radionuclides will not reach the tumor.

In some embodiments, the activity on the sheet is selected according to the properties of the gel, to ensure that sufficient radiation reaches the tumor. For example, for a thicker layer of gel, a higher level of activity is used. In some embodiments, the gel is formed from a material which allows a high level of diffusion of lead-212. In some embodiments, the gel includes small protein molecules which have a very strong affinity to lead, such that the lead and small molecule together have a diffusion coefficient of at least 5·10⁻⁸.cm²/sec, at least 2·10⁻⁷.cm²/sec, or even at least 1·10⁻⁶.cm²/sec. Examples include ceruloplasmin (molecular weight 132 kDa), albumin (66 kDa), globulins (~10-60 kDa), azu-globulin (19 kDa), acyl-CoA (9 kDa), and thymosin ß4 (5 kDa). The number density of the small protein molecules in the gel is of the order of the number density of the radium-224 in the sheet. Alternatively, the number density of the small protein molecules in the gel is substantially greater (e.g., at least five times greater or even at least ten times greater) than the number density of Lead-212 on the sheet surface, in order to ensure that a large percentage of the radium-224 and/or Lead-212 binds to the small protein molecules.

Referring in more detail to placing (510) the protective mask, in some embodiments, contours of the tumor to be treated are determined and a mask in the shape of the tumor is created. The mask allows passage of diffusing atoms, alpha radiation and optionally also beta radiation to the tumor and blocks diffusing atoms and optionally also alpha radiation from reaching the patient's skin or other tissue beyond the tumor. The mask may include, for example, a thin metal foil with an aperture matching the contours of the tumor. In some embodiments, the mask is provided with gel in the aperture. In treatment, the mask is placed on the patient in a manner which matches the contours of the tumor and the sheet with the radium radionuclides is placed above the mask and gel. Alternatively, the radium is coupled to the radiotherapy sheet in an area which matches the contours of the tumor.

It is noted that the method of FIG. 5 includes various acts which are optional and are not necessarily included in all embodiments. In some embodiments, application of the radiotherapy sheet includes a single step of pacing the sheet on a tissue surface requiring treatment.

It is noted that some of the above-described embodiments may include structure, acts or details of structures and acts that may not be essential to the invention and which are described as examples. Structure and acts described herein are replaceable by equivalents which perform the same function, even if the structure or acts are different, as known in the art. The embodiments described above are cited by way of example, and the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art. Therefore, the scope of the invention is limited only by the elements and limitations as used in the claims, wherein the terms “comprise,” “include,” “have” and their conjugates, shall mean, when used in the claims, “including but not necessarily limited to.”

1. A sheet for radiotherapy, comprising:
a sheet base configured for placement on tissue of a patient;
a radium-binding material coupled to the base sheet; and
alpha-emitting radium radionuclides coupled to the radium-binding material, wherein the radium radionuclides do not leave the sheet, but radon and lead resulting from decay of the radium radionuclides, easily leave the sheet.

2. The sheet of claim 1, wherein the sheet base is formed from the radium-binding material.

3. The sheet of claim 1, wherein the sheet base is formed of a material different from the radium-binding material.

4. The sheet of claim 3, wherein the radium-binding material is coated over the sheet base.

5. The sheet of claim 3, wherein the radium-binding material comprises mini-particles dispersed on the sheet base.

6. The sheet of claim 3, wherein the radium-binding material comprises micro-particles dispersed on the sheet base.

7. The sheet of claim 3, wherein the radium-binding material comprises nano-particles dispersed on the sheet base.

8. The sheet of claim 1, wherein the alpha-emitting radium radionuclides have an activity of at least 1 microcurie per centimeter length.

9. The sheet of claim 1, wherein the radium-binding material comprises manganese oxide.

10. The sheet of claim 1, wherein the radium-binding material comprises alginate.

11. The sheet of claim 1, wherein the sheet base comprises a silicone substrate.

12. The sheet of any of claims 1-11, wherein the sheet base comprises a mesh.

13. The sheet of claim 8, wherein the mesh comprises a titanium mesh.

14. A sheet for radiotherapy, comprising:
a sheet base configured for placement on tissue of a patient;
sources of a length of at least one millimeter, carrying alpha-emitting radionuclides with an activity of at least 1 microcurie per centimeter length, coupled to the sheet base, such that at least part of the sources are external to the sheet base; and
17
a layer of gel on the sheet.

15. The sheet of claim 14, wherein the sheet base is configured to mold to contours of the skin of the patient.

16. The sheet of claim 14, wherein the sheet base comprises a silicone sheet.

17. The sheet of claim 14, wherein the sheet base comprises a mesh and the sources have internal tunnels threaded on the mesh.

18. The sheet of claim 14, wherein the sheet base has an area of at least 2 centimeters square.

19. The sheet of claim 14, wherein the alpha-emitting radionuclides comprise radium-224 radionuclides.

20. A sheet for radiotherapy, comprising:
a sheet base configured for placement on tissue of a patient; and
sources of a length of at least one millimeter, carrying alpha-emitting radionuclides with an activity of at least 1 microcurie per centimeter length, coupled to the sheet base, such that at least part of the sources are external to the sheet base, wherein the alpha-emitting radionuclides are coupled to the sources in a manner which prevents detachment of the alpha-emitting radionuclides from the sources but allows escape from the sources of daughter radionuclides of the alpha-emitting radionuclides, upon decay.

21. The sheet of claim 20, wherein the alpha-emitting radionuclides have an activity of at least 5 microcurie per centimeter length.

22. The sheet of claim 20, wherein the sources protrude from the sheet base by at least 1 millimeter.

23. The sheet of claim 20, wherein the sources are coupled to the sheet base by an adhesive.

24. A method of radiotherapy, comprising:
covering a tumor on the skin of a patient with a gel;
providing a sheet to which radium-224 radionuclides at a density of an activity of at least 1 microcurie per square centimeter are coupled; and
placing the sheet on the gel for a duration suitable for radiotherapy.

25. The method of claim 24, wherein the radium-224 radionuclides have an activity of at least 10 microcurie per square centimeter.

26. The method of claim 24, wherein the gel has a thickness of less than 0.6 mm.

27. The method of claim 26, wherein the gel has a thickness of less than 50 microns.

28. The method of claim 26, wherein covering the tumor with the gel comprises placing gel on the sheet and placing the sheet, with the gel thereon, on the skin of the patient.

29. The method of claim 26, wherein placing the sheet on the gel is performed after the tumor is covered by the gel.

30. The method of claim 26, further comprising identifying contours of the tumor and wherein providing the sheet comprises coupling radium-224 radionuclides to the sheet in an area selected responsive to the identified contours, before it is placed on the gel.

31. The method of claim 24, further comprising identifying contours of the tumor and wherein placing the sheet on the gel comprises placing a mask following the contours of the tumor around the tumor and placing the sheet on the gel and the mask.

32. The method of claim 24, wherein the gel does not contain alpha-emitting radionuclides.
